(19) United States
(12) Patent Application Publication
(10) Pub. No.: US 2014/0128864 A1
(43) Pub. Date: May 8, 2014

(54) ELECTROSURGICAL GENERATOR

(71) Applicant: GYRUS MEDICAL LIMITED, Cardiff (GB)

(72) Inventor: Anthony K. ATWELL, Newport (GB)

(21) Appl. No.: 14/069,833

(22) Filed: Nov. 1, 2013

(30) Foreign Application Priority Data
Nov. 8, 2012 (GB) 1220151.3

(51) Int. Cl. A61B 18/18

(52) U.S. Cl. CPC A61B 18/18
USPC 606/37

(57) ABSTRACT

An electrosurgical generator (1) comprises a source of radio frequency (R.F.) energy, at least a pair of output terminals for connection to a bipolar electrosurgical instrument (3) and a pulsing circuit for the source. The generator (1) delivers to the electrosurgical instrument (3) an amplitude-modulated RE power signal in the form of a succession of pulses. The electrosurgical generator (1) has a blended mode comprising a rapidly alternating sequence of a first cutting mode and a second coagulation mode. The first cutting mode comprises pulses (11) having a predetermined voltage amplitude and a first pulse width, and the second coagulation mode comprises pulses (13) having a similar voltage amplitude but a second smaller pulse width.

### TECHNICAL FIELD

This invention relates to an electrosurgical generator for use with electrosurgical instruments for the treatment of tissue. Such instruments are commonly used for the cutting/vaporisation and/or desiccation/coagulation of tissue in surgical intervention, most commonly in “keyhole” or minimally invasive surgery. The terms “cutting” and “vaporization” relate to the removal of tissue, whether by resection or by the volumetric removal of tissue. Similarly, the terms “desiccation” and “coagulation” relate to the creation of lesions in tissue, the necrosis of tissue, and to the prevention of bleeding.

### BACKGROUND TO THE INVENTION AND PRIOR ART

Endoscopic instruments are often used in gastroenterology or cardiac surgery, and such instruments are normally introduced through an endoscope working channel, with the endoscope in turn introduced through a lumen within the patient's body. These instruments are therefore of a relatively small size, often no more than 5 mm in diameter. They are deployed at the end of a relatively long flexible shaft, such that they can be manoeuvred within a lumen as described above.

The electrosurgical waveforms used to drive endoscopic instruments must be such as to allow the instrument to perform the cutting or coagulation of tissue, even though the end effector at the tip of the endoscopic instrument is of a relatively small size. The present invention attempts to provide an improvement to electrosurgical generators for use with endoscopic instruments of this type.

### SUMMARY OF THE INVENTION

Accordingly, an electrosurgical generator is provided comprising a source of radio frequency (r.f.) energy, at least a pair of output terminals for connection to a bipolar electrosurgical instrument and for delivering r.f. energy from the source to the instrument, and a pulsing circuit for the source, wherein the pulsing circuit and the source are so arranged as to deliver into a resistive load, when connected across the output terminals, an amplitude-modulated r.f. power signal in the form of a succession of pulses, the electrosurgical generator having a blended mode comprising a rapidly alternating sequence of a first cutting mode and a second coagulation mode, the first cutting mode comprising pulses having a predetermined voltage amplitude and a first pulse width, and the second coagulation mode comprising pulses having a similar voltage amplitude but a second smaller pulse width.

In general, electrosurgical generators deliver a continuous burst of radio frequency energy, either at a relatively high amplitude in order to cut tissue, or at a relatively lower amplitude in order to coagulate tissue. By “amplitude” is meant the level of energy delivered, whether the absolute level of the voltage or the current is varied. Typically, a voltage of 300V-450V may be used for tissue cutting, while a voltage of 50-250V is used for tissue coagulation.

It is known to provide a modulated RE waveform consisting of pulses of RF energy, typically for the coagulation of tissue. U.S. Pat. No. 6,893,435 is an example of this type of electrosurgical generator. It is also known to provide a continuous RE electrosurgical cutting waveform, and to use pulses of the continuous waveform as a coagulating waveform. U.S. Pat. No. 3,885,569 is an example of this type of electrosurgical generator. The present invention is different from either of these prior art systems. RF cutting is achieved using pulses of a certain amplitude and having a certain pulse width. RE coagulation is achieved by using pulses of a similar amplitude but with a shorter pulse width. Furthermore, a blended mode is available with a rapid alternating of bursts of the shorter and longer pulses. Embodiments of the present invention therefore modulate the pulse width of an otherwise substantially constant envelope signal in order to achieve cutting or coagulation modes of operation, with the pulse width required for cutting mode being longer in time than the pulse width required for coagulation mode.

The pulses in the first and/or second modes are separated with periods of reduced voltage, typically less than 50V, conveniently less than 5V and preferably substantially zero. The predetermined voltage amplitude is typically greater than 200V rms, conveniently between 300V and 400V rms, and preferably between 320V and 360V rms. typically 340V rms. The first cutting mode typically comprises pulses of a pulse width of between 1 ms and 10 ms, conveniently between 2 ms and 6 ms, and preferably approximately 3 ms. The second coagulating mode typically comprises pulses of a pulse width of between 0.5 ms and 3 ms, conveniently between 1 ms and 2 ms, and preferably approximately 1.5 ms. Although the above figures are typical examples, there are various factors that will determine whether a particular RE voltage will act in a tissue-cutting or tissue-coagulating fashion, including the voltage amplitude, pulse width, the period between the pulses, the design of the electrode, the characteristics of the tissue and many others. In general terms, the pulses in the first RE mode are sufficient to cause the cutting of tissue, and the pulses in the second RE mode are insufficient to cause the cutting of tissue such that they merely cause the tissue to become coagulated.

In one convenient arrangement, the pulsing circuit is arranged to vary the pulse width such as to cause a smooth transition between the first and second modes. In this way, the pulse width does not change abruptly from the pulse width associated with the first mode to that associated with the second mode. Instead, the pulse width varies gradually, typically over a period of 3 or more pulses, between the first and second modes. For example, the pulse width may linearly interpolate over a number of pulses. such as a predefined number typically 3 or more, between the pulse width associated with the first mode and the pulse width associated with the second mode. Likewise, when switching from the second mode to the first mode, the pulse width may be modulated to cause a smooth transition. The modulation may be the reverse of when switching from the first more to the second mode i.e. the pulse width is interpolated over a number of pulses, typically 3 or more, between the pulse width associated with the second mode and the pulse width associated with the first modem n another convenient arrangement, the generator is able to measure a parameter associated with a surgical procedure, and, when operated in the blended mode, to vary the proportion of the first cutting mode as compared with the second coagulation mode depending on the measured parameter. In this way, the generator can react to different situations, such as different tissue characteristics, by changing the proportion of the cutting or coagulation waveforms provided. In one convenient arrangement, the measured parameter is tissue impedance. Typically, if the tissue impedance is high, indicating a relatively dry tissue environment, the generator can supply more of the tissue-cutting component of the blended signal. Conversely, if the tissue impedance is low, indicating a relatively moist tissue environment associated with the presence of blood and other fluids, the generator can supply more of the tissue-coagulating component of the blended signal. The generator is therefore able to react to the changing circumstances, and provide a waveform most suited to the needs of the operator.

As well as operating in the blended mode, in some embodiments the generator is able to operate separately solely in either the first cutting mode, or the second coagulation mode. In such embodiments the generator provides either the first cutting mode signal or the second coagulation mode signal as an output when activated, and does not automatically switch therebetween. Controls can be provided on the generator or on the instrument to allow for selection of the desired mode i.e. cutting, coagulation, or blended. In this way, the operator can control the operation of the generator to in provide a blended cutting and coagulation signal, or a cutting only signal, or a coagulation only signal.

From another aspect the present invention also relates to a method of operating an electrosurgical generator, comprising: delivering an RF power signal in the form of a succession of pulses of substantially constant amplitude from at least a pair of output terminals to a bipolar electrosurgical instrument, and pulse-width modulating the succession of pulses to provide a blended mode of operation which both cuts and coagulates tissue during operation in the blended mode. Preferably the pulse width modulating comprises time-dividing, the RF power signal into a first period during which pulses having a first pulse width corresponding to a cutting mode of operation are output and a second period during which pulses having a second, shorter, pulse width corresponding to a coagulation mode of operation are output, the first and second periods automatically alternating during the blended mode of operation.

A further aspect of the present invention also provides an electrosurgical generator, comprising: a radio frequency (RF) source arranged to output an RF signal; a pulse width modulator arranged to convert the RF signal into a succession of pulses of substantially constant amplitude and varying width; and a pair of output terminals arranged in use to supply the succession of pulses to a bipolar electrosurgical instrument, wherein the pulse width modulator is further arranged to pulse width modulate the succession of pulses to provide a blended mode of operation which both cuts and coagulates tissue during operation in the blended mode.

In one embodiment the pulse width modulator is preferably further arranged to time-divide the RF signal into a first period during which pulses having a first pulse width corresponding to a cutting mode of operation are output and a second period during which pulses having a second, shorter, pulse width corresponding to a coagulation mode of operation are output, the first and second periods automatically alternating during the blended mode of operation.

### BRIEF DESCRIPTION OF THE DRAWINGS

Embodiments of the invention will now be further described, by way of example only, with reference to the accompanying drawings, in which:

FIG. 1 is a schematic diagram of an electrosurgical system employing a generator according to the present invention,

FIGS. 2A & 2B are schematic diagrams of waveforms produced by the generator of FIG. 1,

FIGS. 3A & 3B are schematic diagrams of alternative waveforms produced by the generator of FIG. 1,

FIGS. 4A & 4B are schematic diagrams of further waveforms produced by the generator of FIG. 1, and

FIG. 5 is a schematic diagram of a further waveform produced by the generator of FIG. 1.

### DESCRIPTION OF THE EMBODIMENTS

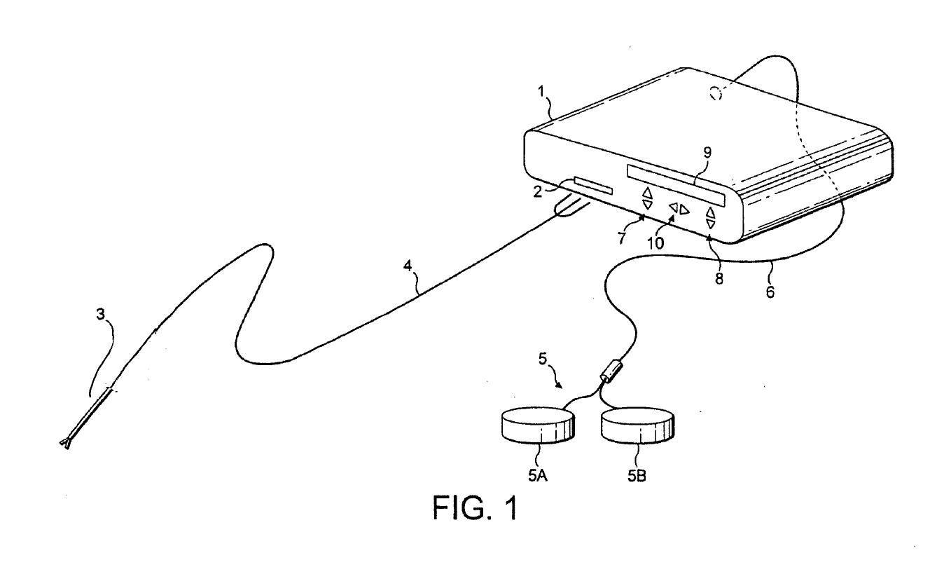

Referring to FIG. 1, a generator 1 has art output socket 2 providing a radio frequency (RF) output for an instrument 3 via a connection cord 4. Activation of the generator I may be performed from the instrument 3 via a connection in the cord 4, or by means of a footswitch unit 5, as shown, connected to the rear of the generator by a footswitch connection cord 6. In the illustrated embodiment, the footswitch unit 5 has two footswitches 5A and 5B for selecting a coagulation mode and a cutting mode of the generator 1 respectively. The generator front panel has push buttons 7 and 8 for respectively setting coagulation and cutting power levels, which are indicated in a display 9. Push buttons 10 are provided as an alternative means for selection between coagulation and cutting modes.

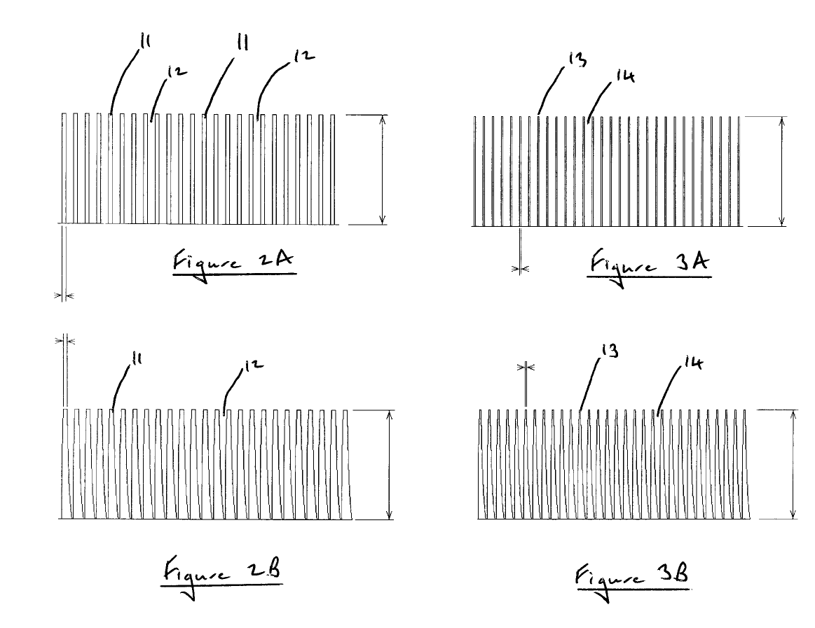

FIGS. 2A & 2B show the waveform produced by the generator 1 when the cutting mode is selected, either from the footswitch unit 5 or from the push buttons 10. FIG. 2A shows an idealised waveform in which pulses 11 of amplitude-modulated RF energy are provided to the instrument 3. The pulses are of a voltage of approximately 450V, and are approximately 3 ms in width. Between the pulses 11 are periods 12 of substantially zero voltage, the “off” periods being substantially 7ms in duration. FIG. 2B shows a non-idealised waveform which is more likely to depict the real waveform provided to the instrument 3. The pulses 11 have a rapid but non-instantaneous rise due to the rise time of the generator 1, and a non-instantaneous drop in voltage as the voltage decays.

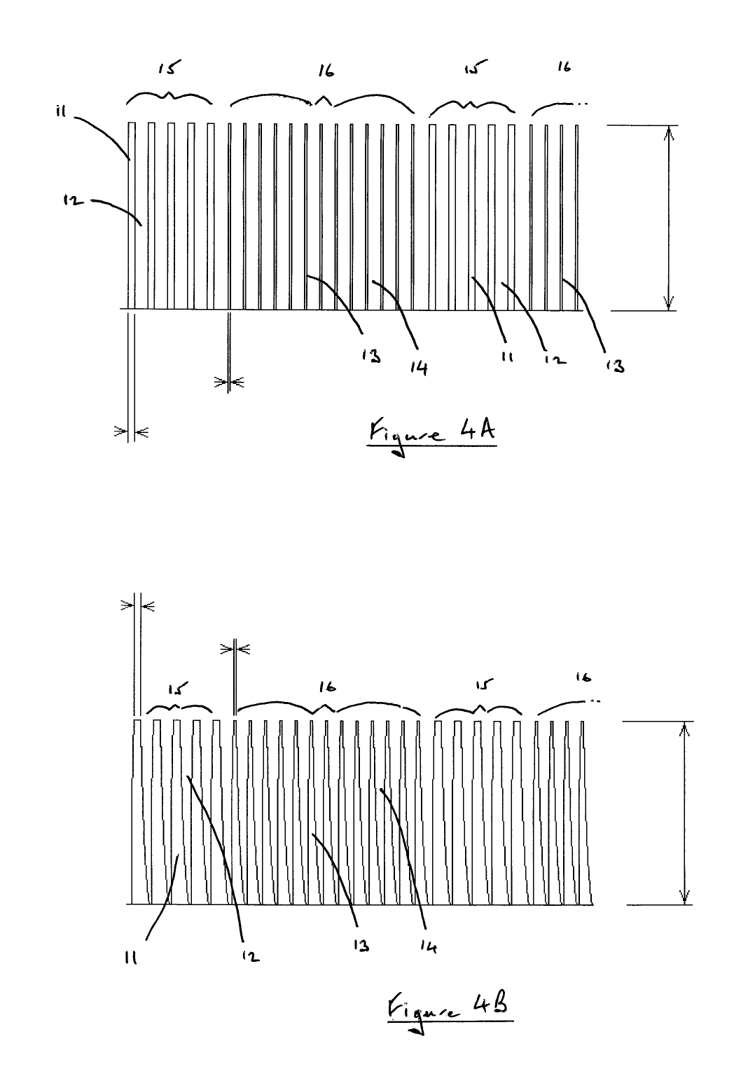

FIG. 3A & 3B show the waveform produced by the generator 1 when the coagulation mode is selected, again either from the footswitch unit 5 or from the push buttons 10. FIG. 3A shows the idealised waveform in which pulses 13 of amplitude-modulated RF energy are provided to the instrument 3. The pulses are once again of a voltage of approximately 450V, but this time are no more than approximately 1 ms in width. Between the pulses 13 are periods 14 of substantially zero voltage, the “off” periods being substantially 9 ms in duration. Despite the pulses 13 being of the same peak voltage as those of the cutting mode, the decreased pulse width and the increased “off” period ensures that the instrument 3 does not fire up into cutting mode and cause tissue vaporisation, but remains in a sub-vaporisation mode able to coagulate tissue in contact therewith. FIG. 3B once again shows a non-idealised waveform which is more likely to depict the real waveform provided to the instrument 3.

Comparing FIGS. 3A and 3B relating to the coagulation mode with FIGS. 2A and 2B relating to the cutting mode, it will be seen that selection between the cutting mode and coagulation mode is thus achieved by modulating the pulse width between the two modes, in that the pulse width for the cutting mode is longer than the pulse width for the coagulation mode.

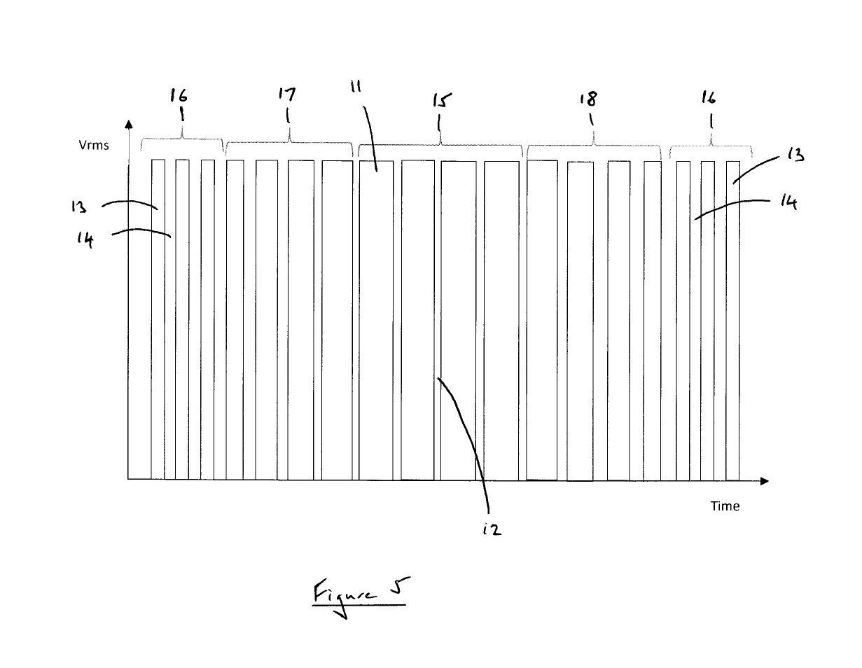

FIGS. 4A & 4B show a blended mode, once again selected either from the footswitch unit 5 or from the push buttons 10. In this blended mode, the waveform supplied by the generator 1 to the instrument 3 consists of periods 15 of the cutting mode including the pulses 11 and “off” periods 12, alternating with periods 16 of the coagulation mode including the pulses 13 and “off” periods 14. FIG. 4B once again shows a non-idealised waveform which is more likely to depict the real waveform provided to the instrument 3. In this blended mode, the periods 15 cause vaporisation of tissue in contact with the instrument 3, while the periods 16 cause tissue in contact with the instrument 3 to be coagulated. As the periods 15 and 16 alternate repeatedly and very rapidly, the overall effect is that the tissue is both cut and coagulated simultaneously.

FIG. 5 shows an idealised version of a blended mode in which there is no abrupt transition between the periods 15 and 16, but in which the waveform transitions therebetween over a period of several pulses. In FIG. 5 the coagulation period is once again shown at 16 and consists of pulses 13 and “off” periods 14. The cutting period is shown at 15, and consists of pulses 11 and “off” periods 12. In between the periods 16 & 15 is a transition period 17 in which the pulse width gradually increases from the 1 ms pulse width of the pulses 13 to the 3 ms pulse width of the pulses 11. This takes place over 4 pulses in FIG. 5. After the cutting period 15, the generator switches back to the coagulating period 16 byway of a further transition period 18, in which the pulses decrease from the 3 ms pulse width of the pulses 11 to the lens pulse width of the pulses 13. This again takes place over 4 pulses as shown in FIG. 5. The blended waveform therefore consists of a repeating sequence of coagulating period 16, transition period 17, cutting period 15, transition period 18, coagulating period 16 etc. etc.

In FIGS. 4A & 4B, the duration of the cutting period 15 is 5 pulses and the duration of the coagulating period 16 is 13 pulses. These durations can be changed by selecting alternative profiles on the generator 1. Alternatively, the durations of the cutting and coagulating periods can be adjusted automatically in response to feedback to the generator 1 from the instrument 3. For example, the generator may monitor changing values for the current supplied to the instrument in order to establish a measurement of the impedance of the tissue in contact with the instrument. Depending on the measured tissue impedance, the generator may automatically adjust the cutting and coagulating periods respectively. In one situation, here the current being supplied is relatively low, indicating that the tissue is exhibiting a relatively high impedance, probably because it is relatively dry, the generator automatically increases the number of pulses in the cutting period 15. In this situation, the cutting period 15 is typically 10 pulses in length, while the coagulating period 16 is 8 pulses in length. Alternatively, where the current being supplied is relatively high, indicating that the tissue is exhibiting a relatively low impedance, probably because it is relatively moist indicating the presence of bleeding, the generator automatically decreases the number of pulses in the cutting period 15 and increases the number of pulses in the coagulating period 16. In this situation, the cutting period 15 is typically 5 pulses in length, while the coagulating period 16 is 13 pulses in length (as shown in FIG. 4A & 4B). In this way, the generator is able to adjust the tissue effect achieved by the instrument 3, depending on what is required as indicated by the measured tissue impedance.

Other variations can be envisaged without departing from the scope of the present invention. For example, a variety of different pulse widths, pulse lengths and periods between pulses can be provided, as can the parameters used to provide feedback for the control of the blended mode. It will be appreciated that the key elements of the invention are the use of cutting and coagulating pulses of similar amplitude but different pulse duration, together with a blended mode consisting of combinations of both waveforms. The generator may conveniently be selected to deliver any of the cutting mode, the coagulation mode or the blended mode as required. Conceivably, the generator only delivers the blended mode, as this performs both a cutting and a coagulating action.

1. An electrosurgical generator comprising a source of radio frequency (r.f.) energy, at least a pair of output terminals for connection to a bipolar electrosurgical instrument and for delivering, r.f. energy from the source to the instrument, and a pulsing circuit for the source, wherein the pulsing circuit and the source are so arranged as to deliver an r.f. power signal in the form of a succession of pulses, the electrosurgical generator having a blended mode comprising, a rapidly alternating sequence of a first cutting mode and a second coagulation mode, the first cutting mode comprising pulses having a predetermined voltage amplitude and a first pulse width, and the second coagulation mode comprising pulses having a similar voltage amplitude but a second smaller pulse width.

2. An electrosurgical generator according to claim 1, wherein the pulses in the first cutting mode are separated by periods in which the voltage is less than 50V, or more preferably less than 5V or even more preferably substantially zero.

3. An electrosurgical generator according to claim 1, wherein the pulses in the second coagulation mode are separated by periods in which the voltage is less than 50V, or more preferably less than 5V, or even more preferably substantially zero.

4. An electrosurgical generator according to claim 1, wherein the predetermined voltage amplitude is greater than 200V nits, or more preferably between 300V and 400V rms, or even more preferably between 320V and 360V rms.

5. An electrosurgical generator according to claim 4, wherein the predetermined voltage amplitude is approximately 340V rms.

6. An electrosurgical generator according to claim 1, wherein the first cutting mode comprises pulses of a pulse width of between 1 ms and 10 ms, or more preferably between 2 ms and 6 ms, or even more preferably of approximately 3 ms.

7. An electrosurgical generator according to claim 1 wherein the second coagulating mode comprises pulses of a pulse width of between 0.5 ms and 3 ms, or more preferably of between 1 ms and 2 ms, or even more preferably of approximately 1.5 ms.

8. An electrosurgical generator according to claim 1, wherein the pulsing circuit is arranged to vary the pulse width such as to cause a smooth transition between the first and second modes.

9. An electrosurgical generator according to claim 8, wherein the pulsing circuit is arranged to transition between the first and second modes over a period of 3 or more pulses.

10. An electrosurgical generator according to claim 9, wherein the pulsing circuit is arranged to interpolate between the pulse widths of the first and second modes for the pulse widths of the pulses in the period.

11. An electrosurgical venerator according to claim 1, wherein the venerator is arranged to measure a parameter associated with a surgical procedure, and, when operated in the blended mode, to vary the proportion of the first cutting mode as compared with the second coagulation mode depending on the measured parameter.

12. An electrosurgical generator according to claim 11, wherein the measured parameter is tissue impedance.

13. An electrosurgical generator according to claim 1, and further arranged to be capable of operating solely in either the cutting mode or the coagulation mode.

14. A method of operating an electrosurgical generator, comprising:
delivering an RF power signal in the form of a succession of pulses of substantially constant amplitude from at least a pair of output terminals to a bipolar electrosurgical instrument; and
pulse-width modulating the succession of pulses to provide a blended mode of operation which both cuts and coagulates tissue during operation in the blended mode.

15. A method according to claim 14, wherein the pulse width modulating comprises time-dividing the RF power signal into a first period during which pulses having a first pulse width corresponding to a cutting mode of operation are output and a second period during which pulses having a second, shorter, pulse width corresponding to a coagulation mode of operation are output, the first and second periods automatically alternating during the blended mode of operation.

16. An electrosurgical generator, comprising:
a radio frequency (RF) source arranged to output tan RF signal;
a pulse width modulator arranged to convert the RF signal into a succession of pulses of substantially constant amplitude and varying width; and
a pair of output terminals arranged in use to supply the succession of pulses to a bipolar electrosurgical instrument
wherein the pulse width modulator is further arranged to pulse width modulate the succession of pulses to provide a blended mode of operation which both cuts and coagulates tissue during operation in the blended mode.

17. An electrosurgical generator according to claim 16, wherein the pulse width modulator is further arranged to time-divide the RF signal into a first period during which pulses having a first pulse width corresponding to a cutting mode of operation are output and a second period during which pulses having a second, shorter, pulse width corresponding to a coagulation mode of operation are output, the first and second periods automatically alternating during the blended mode of operation.
